(19) United States
(12) Patent Application Publication
(10) Pub. No.: US 2017/0325818 A1
(43) Pub. Date: Nov. 16, 2017

(54) Novel Vascular Clamp

(71) Applicant: Dean Trivisani, Oakland, NJ (US)

(72) Inventor: Dean Trivisani, Oakland, NJ (US)

(21) Appl. No.: 15/610,197

(22) Filed: May 31, 2017

Related U.S. Application Data
(63) Continuation of application No. 13/117,192, filed on May 27, 2011, now abandoned.
(60) Provisional application No. 61/349,334, filed on May 28, 2010.

(51) Int. Cl. A61B 17/122

(52) U.S. Cl. CPC A61B 17/122; A61B 17/1227

(57) ABSTRACT

The present invention relates to vascular clamps, in particular, a clamp designed to clamp a vein or artery such as an umbilical vascular, without severing the vasculature.

### CLAIM OF PRIORITY

This application is a continuation of U.S. application Ser. No. 13/117,192 filed on May 27, 2011 which claims priority to U.S. Application 61/349,334, filed on May 28, 2010 both of which are fully incorporated by reference in its entirety.

### FIELD OF THE INVENTION

The invention relates to vascular clamps, in particular, a clamp designed to occlude a vein or artery such as an umbilical cord, without severing the vasculature.

### BACKGROUND OF THE INVENTION

The present invention relates to vascular clamps, in particular, a clamp designed to occlude a vein or artery such as an umbilical cord, without severing the vasculature. This invention offers multiple improvements over those know in the art. First, the width of the finger grip is enlarged to accommodate larger hands or hands with gloves. Second, this widening prevents the user's fingers from slipping off the vascular clamp when closing the clamp because of fluids making the gloves slimy. Third, there are larger teeth which permit improved grasping of the umbilical cord. Fourth, the increased length of the device provides better accommodation for electronic devices which may be optionally attached. Further, the increased length better accommodates thicker umbilical cords, and accordingly, provides for easier closure of the device.

General hospital-based obstetric practice introduces artificial clamping as early as 1 minute after the birth of the child. Clamping is followed by cutting of the vascular, which is painless due to the lack of any nerves. The vascular is extremely tough, like thick sinew, and so cutting it requires a suitably sharp instrument. Provided that umbilical severance occurs after the vascular has stopped pulsing (5-20 minutes after birth), there is ordinarily no significant loss of either venous or arterial blood while cutting the vascular.

After the vascular is clamped and cut, the newborn wears a plastic clip on the navel area until the compressed region of the vascular has dried and is sealed sufficiently. The remaining umbilical stub remains for up to 7-10 days as it dries and then falls off.

U.S. Patent Application No. 20040199178 is directed to a releasably locking umbilical cord clamp including one or more of: a gender-identifying color, a grasping portion to facilitate closing and locking the clamp on the remaining umbilical cord, a fluid removal channel, a separable measurement portion to facilitate positioning of the clamp at a suitable location on an umbilical cord, and an operatively associated key to unlock and reposition or remove the clamp. Also included are methods relating to using a gender-identifying color on an umbilical cord clamp to facilitate gender and identity recognition and methods of applying the clamp at the proper distance. The length is 1 to 4 cm.

U.S. Pat. No. 3,247,852 discloses an inexpensive, disposable, and widely used umbilical cord clamp for closing the umbilical cord of a newborn infant. The clamp is formed of flexible plastic, has a pair of arms joined by an integral hinge, and is provided with locking means in the form of a hook portion receivable in a recess when the clamp is closed.

U.S. Pat. No. 4,212,303 discloses a V-shaped umbilical cord clamp having a lock construction of a flexible tongue on one arm and a forwardly-facing recess for receiving the tongue as the clamp is closed. A pair of projections extend inwardly from opposite sides of the recess and define sloping ramp surfaces for engaging the tip of the tongue and for flexing the tongue forwardly as the arms of the clamp are squeezed into the closed position.

U.S. Pat. No. 4,428,374 discloses a pair of spaced apart umbilical cord clamping members and a tool for closing and cutting the same. The tool locks when closed and must be deactivated to open the tool.

U.S. Pat. No. 5,423,831 discloses a plastic umbilical cord clamp having a tongue with a hook on one arm and a body with a transverse groove on the other arm for engaging with the hook.

U.S. Pat. No. 5,006,830 discloses a conventional umbilical cord clamp including an identification system to deter the unauthorized removal of a newborn from a defined area. The system uses identification marks including a serial code, bar code, color code, or letter combination, each of which has a different distinctive mark thereon. Merritt also teaches a groove present in a portion of each arm along a portion of the length of each arm to prevent the clamp from slipping off the umbilical cord.

U.S. Pat. No. 5,512,879 discloses a miniature electronic security tag affixed to the ankle of newborn infants that contains an RF transmitter and a digital encoding circuit. The tag permits continuous monitoring to alert a central monitoring computer if the tag is cut or stretched or if an unauthorized person attempts to leave the hospital with the infant.

U.S. Pat. No. 5,608,382 discloses an infant identification and security system including an umbilical cord clamp and a matching wristband for the infant's mother, with a pair of information storage modules attached to the clamp and wristband and a compatible terminal for reading and writing information thereto. Triggering elements are included for triggering a compatible alarm system if an unauthorized person removes the newborn from the maternity ward.

U.S. Pat. No. 5,921,991 discloses umbilical cord clamps having two or more different colors at the distal end of each elongated arm of the clamps. The colors are complementary such that, when combined, they create a third color to indicate that the clamp is closed.

U.S. Pat. No. 5,968,054 discloses a device and method for clamping and severing a compressible structure containing fluid. A clamp having a pair of arms is secured in a clamped position while forcing fluid outwardly therefrom, and a cutting assembly cuts through the pair of arms to form separated first and second clamps.

U.S. Pat. No. 5,938,666 discloses a unitary clamp structured from two U-shaped members disposed one from the other by an interjoining web between proximate arms of each member. The web sheets can later be cut and the clamps closer to the placenta can be removed or discarded.

U.S. Pat. No. 6,212,808 discloses a safety identification assembly for use in neonatology including identifying sub-assemblies detachably connected to each other for identifying mother and baby and for closing umbilical cord ends.

U.S. Pat. No. 5,462,555 is directed to a surgical instrument for clamping a maternal side of an umbilical cord, applying an umbilical cord clip to a fetal side of the umbilical cord and severing the cord intermediate the clamped maternal and fetal sides of the umbilical cord. The invention provides a recessed severing mechanism that is partially moveable to severe the umbilical cord. It further has an attachment means on at least one of the sides to hold the umbilical cord clip on an applicator. The arms are 10 cm long and 2.5 cm wide

U.S. Pat. No. 5,279,915 is directed to a surgical instrument for severing the umbilical cord wherein one of the jaws is a demountable cutting blade. It includes a clamp having a pair of arms joined together at an integral hinge and head portions including locking means at the free ends thereof is provided with a channel or opening extending generally diagonally through at least one of the head portions, such channel communicating with the locking means in the head portion to release such locking means when the arms are locked together in clamping position. The locking mechanism is a tongue which is released with a separate instrument.

The prior art has numerous defects which are solved by the current invention. For instance, the previous designs are manufactured in such a way that the clamp often does not remain closed. This is directly related to the manufacturing style, which we have solved.

One clamp (U.S. application Ser. No. 12/658,622) is designed with a substantially raised bar between the teeth of the clams. These small teeth coupled with this design effectively almost always cause the clamp to slip off the umbilical vascular. This same design defect has also been known to pinch the vascular too much to the point where the clamp severs the umbilical vascular. In addition, the small grip makes the device difficult to use and it often slips from hands wet from fluids.

Release of or premature severance of the umbilical cord can lead to cardiac asystole and hypovolemic shock in the neonate. Clinicians have hypothesized that the occurrence of sudden cardiac asystole at birth is due to extreme hypovolemic shock secondary to the loss of blood. At birth, the sudden release of pressure on the infant's body results in hypoperfusion resulting in low central circulation and blood pressure. Severe hypovolemic shock from these effects leads to sudden cardiac arrest. Immediate vascular clamping maintains the hypovolemic state by preventing the physiologic and readily available placental blood from returning to the infant. Loss of this blood initiates an inflammatory response leading to seizures, hypoxic-ischemic encephalopathy, and brain damage or death. Animal studies have shown that human umbilical stem cells injected into a rat's abdomen after induced brain damage, can protect the rat's brain from developing permanent injury. To prevent damage to newborns, the infant must receive the blood volume and stem cells lost at the time of descent and immediate vascular clamping. [Med Hypotheses. 2009 April; 72(4):458-63. Epub 2009 Jan. 1. Cardiac asystole at birth: Is hypovolemic shock the cause?

Tetanus caused by Clostridium sp. is a common cause of infant mortality in the developing world. Tetanus is now a rare disease in developed world. However it remains an important cause of death worldwide and is associated with a high case fatality, particularly in the developing world. Tetanus is caused by contamination of wound by spores of Clostridium tetani. Neonatal tetanus results from contamination of the umbilical stump at or following delivery of a child born to a mother who did not possess sufficient circulatory antitoxin to protect the infant passively by transplacental transfer. It produces its clinical effects via a powerful exotoxin, tetanospasmin, which leads to uncontrolled disinhibited efferent discharges from motor neurons in the spinal vascular and brainstem, causing intense muscular rigidity and spasm. Shorter incubation and onset times are associated with more severe disease and poorer prognosis. Four clinical forms of tetanus are recognized. They are generalized, localized, cephalic and neonatal tetanus. Tetanus is associated with several complications like respiratory failure, cardiovascular instability, renal failure and autonomic dysfunctions. Recovery from tetanus usually requires an extended period of time. [Kathmandu Univ Med J (KUMJ). 2009 July-September; 7(27):315-22. Tetanus. Poudel P, Budhathoki S, Manandhar S. (ABSTRACT)]

Accordingly, there remains the possibility that in certain instances, neonatal mortality from infection or cardiac arrest may result where the umbilical cord is not properly clamped or if the clamp does not remain in place, especially early on. The new invention contained herein solves this issue.

The embodiments of this invention are illustrated in the accompanying drawings and will be described in more detail herein below.

### SUMMARY OF THE INVENTION

The invention relates to a vascular clamp comprising a clamp top, a clamp bottom, a locking mechanism, a top jaw, a top jaw riser, a bottom jaw, a bottom jaw riser, a top grip, a bottom grip, teeth and a hinge, and wherein said top grip and said bottom grip are each about 9.4 mm in length and about 9.4 mm in width.

It is an object of this invention to provide an inexpensive device that easily attaches to a neonate's umbilical cord and does not subsequently fall off.

It is another object of this invention to provide a device that is easy to place on the cord despite the presence of birthing fluid or the wearing of gloves by the health care professional.

### BRIEF DESCRIPTION OF THE DRAWINGS

FIG. 1 is a perspective view of a preferred embodiment of the invention in an open position.

FIG. 2 is a perspective view of a preferred embodiment of the invention in a closed position.

FIG. 3 is a left side view of a preferred embodiment of the invention.

FIG. 4 is a right side view of a preferred embodiment of the invention.

FIG. 5 is a top view of a preferred embodiment of the invention.

FIG. 6 is a bottom view of a preferred embodiment of the invention.

FIG. 7 is a front view of a preferred embodiment of the invention.

FIG. 8 is a back view of a preferred embodiment of the invention.

FIG. 9 is a perspective view of a preferred embodiment of the invention.

FIG. 10 is a perspective view of a preferred embodiment of the invention.

### DESCRIPTION OF THE PREFERRED EMBODIMENTS

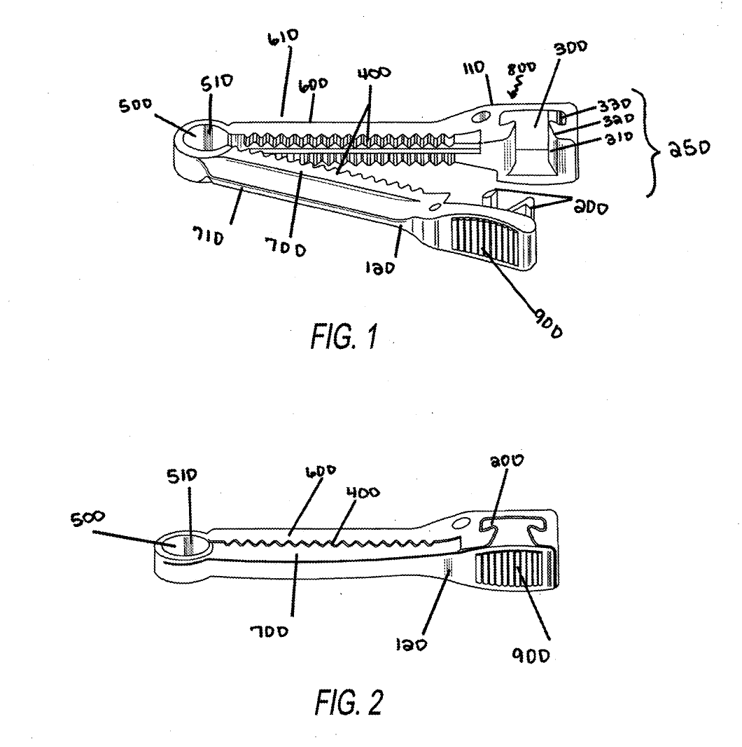

The preferred embodiments of the present invention will now be described with reference to FIG. 1-10 of the drawings. Identical elements in the various figures are identified with the same reference numerals.

FIG. 1 shows a vascular clamp 100, clamp top 110, clamp bottom 120, a top jaw 600, a top jaw riser 610, a bottom jaw 700, a bottom jaw riser 710, a hinge 500, an opening 510, a locking mechanism 210, hooks 200, and a receptacle 300, a top grip 800 and a bottom grip 900. In a preferred embodiment, the top jaw 600 and the bottom jaw 700, are moveably connected at the hinge 500. In a preferred embodiment, the clamp top 110 and the clamp bottom 120 are moveably connected at the hinge 500.

The vascular clamp 100 may be manufactured with the jaws, top jaw 600 and bottom jaw 700, as an integral device or they may be fused together during manufacturing. The teeth 400 are placed around the umbilical vascular of a neonate just before the umbilical vascular is cut off from the placenta and is used to stop the bleeding and prevent infection in the remaining stump. The opening 510 is optional and is present as a backward compatibility feature, to accommodate the wires or plug sensors (not shown). In an optional embodiment, the opening 510 may be used to store the radio frequency tag on a ring threaded through the opening 510.

The hinge opening 510 permits tighter locking of the jaws, top jaw 600 and bottom jaw 700. The top jaw 600 and the bottom jaw 700 each extend from the hinge opening 510 and the hinge 500 to the top grip 800 and the bottom grip 900, respectively. Although the hinge 500 is shown as a specific configuration, this is for illustrative purposes and any type of hinge device may be employed.

The preferred method of closing the present invention is utilizing a locking mechanism 250. In the shown embodiment, the locking mechanism consists of hooks 200 which are received by a receptacle 300. The number and complexity of various locking mechanisms 250 and features may vary as long as there is at least one locking mechanism 250 that achieves a permanent coupling.

Still referring to FIG. 1, the locking mechanism 250 is shown having hooks 200, shown as two opposite facing V shaped hooks 200. To operate this locking mechanism 250, V shaped hooks 200 are forced into the receptacle 300 shaped to receive and hold in place the hooks 200. The receptacle has a first opening 310, wherein the walls begin to taper closer together to make a second opening 320 which is narrow and further a wider, third opening 330 is present. In a preferred embodiment, the point where the walls of the receptacle 300 are the closest together forms a 120 degree angle. This 120 degree angle accommodates the hooks 200, which have a span 210 of 40 degrees on the inner side and 100 degrees on the outer side of the span 210.

Once past the narrow point 320, the receptacle 300 opens to a second, wider opening 330 in which the hooks 200 remain in place. In a preferred embodiment, the initial opening may be greater than 4.7 mm. The hooks 200, in a preferred embodiment, are convergent at their origin at the base of the interior side of either the top jaw 600 or the bottom jaw 700, then proceed to diverge. In a preferred embodiment, the hooks may be at their widest divergent point when they are spaced about 5.6 mm to about 8 mm apart.

In another preferred embodiment the top grip 800 and the bottom grip 900 are about 8 mm to about 9.4 mm at their widest point, and the clamp top 110 and clamp bottom 120 are each separately from about 31 mm to about 61 mm in length. At the widest sections each, the top grip 800 and the bottom grip 900 are about 5.3 mm to about 9.4 mm in width.

Additional locking mechanisms may be utilized to provide a supplemental locking strength to the present invention. One skilled in the art will appreciate that other embodiments of permanent locking means are possible and may be used to enable to present invention.

Once the vascular clamp 100 is attached and locked around an umbilical vascular or an umbilical stump of an infant, the teeth 400 make it virtually impossible to remove it without having to cut the clamp 100. The teeth 400 have a serrated edge and, although illustrated as serrated, may be other shapes. The edge may be any shape or functionality that achieves the desired purpose, such as, but not limited to, a flat surface, or an interlocking surface using any shape or pattern.

FIG. 2 discloses the vascular clamp 100 shown in FIG. 1 in a closed position with the top jaw 600 and the bottom jaw 700 and the hinge 500. Also shown in this figure are a clamp top 110, a clamp bottom 120, teeth 400, a hinge opening 510, a clamp locking mechanism 250 and hooks 200 placed in their receptacle 300. The vascular clamp 100 is preferably disposable, but may be reusable.

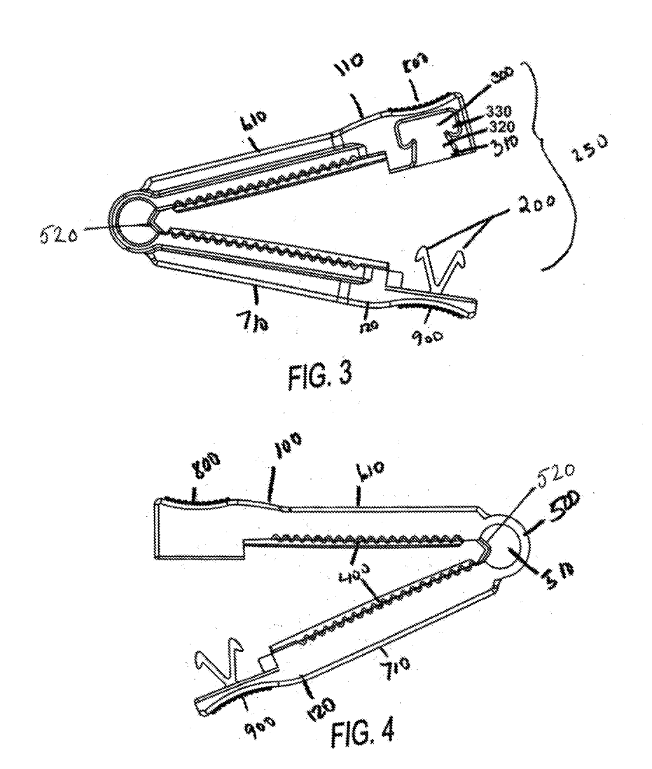

FIGS. 3-10 show alternative embodiments and views of the present invention. Shown in FIGS. 3 and 4 are a vascular clamp 100, top 110, bottom 120, a top jaw 600, a bottom jaw 700, a hinge 500, an opening 510, pins 520, a locking mechanism 250, hooks 200, a receptacle 300, a top grip 800 and a bottom grip 900. Also seen are the top riser 610 and the bottom riser 710. The receptacle 300 may be located wither on the top 110 or the bottom 120 of the vascular clamp. The receptacle 300 has a first opening 310, a second opening 320, and a third opening 330. The hooks 200 may be located either on the top 110 or the bottom 120 of the vascular clamp. According to a preferred embodiment the pins 520 extending from the top and the bottom jaws inside the opening 500 have a spring-like function popping the clamp open when the jaws are not pressed tightly enough to lock the locking mechanism 250.

Note that the top grip 800 and bottom grip 900 are moved to the end of the top and bottom jaws 600 and 700, and are shaped in a way that is convenient for pressing by the human thumb and forefinger. According to a preferred embodiment the top and the bottom grip locate on a concave surface. Further, in a preferred embodiment, the lengths and widths of the top and bottom grips are each about 9.4 mm, that is, their dimensions may be about 9.4 mm by about 9.4 mm. In other embodiments the width is between 5.3 mm to about 9.5 mm.

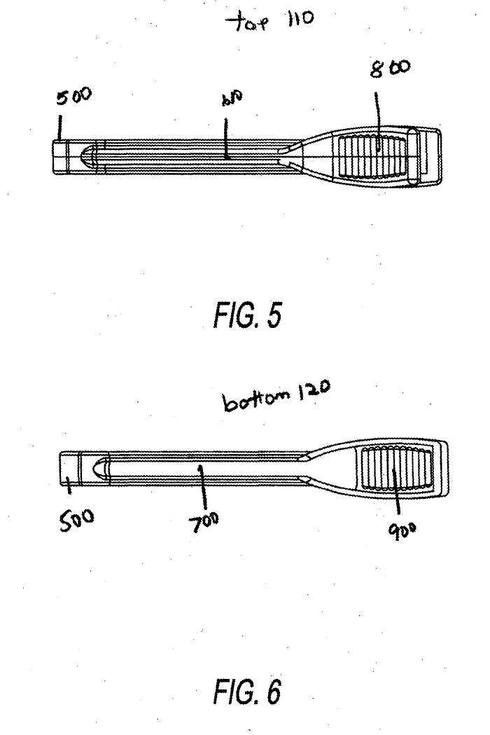

FIG. 5 is a top view of the vascular clamp 100, showing the top of the clamp 110, the top grip 800, the top riser 610 and the hinge 500.

FIG. 6 is a bottom view of the vascular clamp 100, showing the bottom of the clamp 120, the bottom grip 900, the bottom riser 710 and the hinge 500.

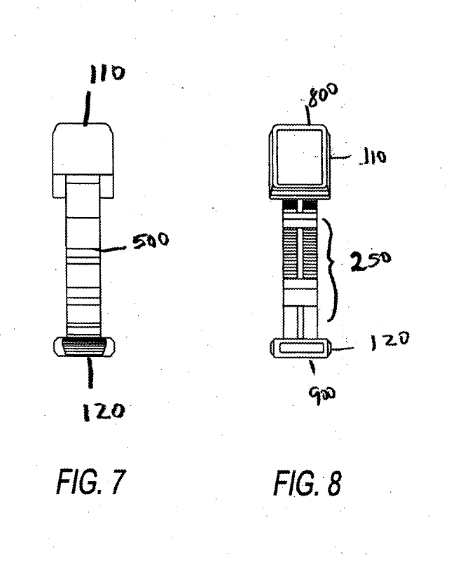

FIG. 7 is a front view of the preferred embodiment of the vascular clamp 100, showing the top of the clamp 110, the hinge 500 and the bottom of the clamp 120.

FIG. 8 is a back view of the preferred embodiment of the vascular clamp 100, showing the top grip 800, the bottom grip 900, the locking mechanism 250 and the bottom of the clamp 120.

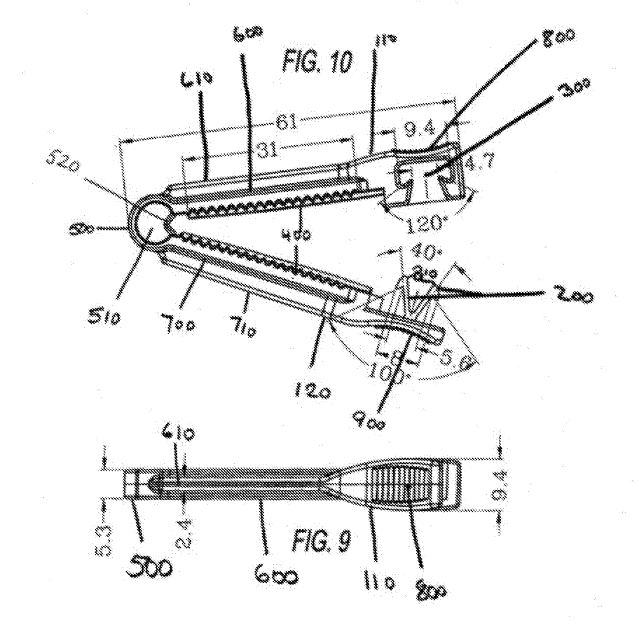

FIGS. 9 and 10 are preferred embodiments of the invention. FIG. 9 is a top view of the vascular clamp 100, showing the top of the clamp 110, the top grip 800, the top riser 610 and the hinge 500. The lengths and the widths of the top grip 800 and the bottom grip 900 are between 5.3 mm to about 9.5 mm. In another embodiment, the top grip 800 and the bottom grip 900 are each about 9.4 mm in length by 9.4 mm in width.

In other embodiments, the top of the clamp 110, and the bottom of the clamp are each about 2 mm to about 10 mm wide. Preferably the clamp top 110, and the clamp bottom 120 are about 2.4 to 5.3 mm wide, more preferably about 9.4 mm wide. It is understood that the lengths or the widths of the clamp top 110, and the clamp bottom 120 can be smaller or larger. Further they can tapper or be same overall lengths and widths. In other cases they may be of different length and different widths.

In one embodiment, the clamp top 110, and the clamp bottom width may start at about 2.4 mm and widen to 5.3 mm, then widen again to 8.4 mm. In a preferred embodiment, the clamp top 110, and the clamp bottom may start at about 5.5 mm wide and expand at the top grip 800 and bottom grip 900 to about 9.4 mm in width.

In other embodiments, the clamp top 100 and the clamp bottom 120 can have the same or different widths.

In a preferred embodiment the top riser 610 and the bottom riser 710 each have a width of about 1 mm to about 3 mm, but are more preferably about 2.4 mm wide.

FIG. 10 is a side perspective of the vascular clamp 100, showing the clamp top 110, the clamp bottom, 120, the v-shaped hooks 200, the hook span 210 and the receptacle 300. The receptacle has a first opening 310, wherein the walls begin to taper closer together to make a second opening 320 which is narrow and further a wider, third opening 330 is present. There is a hinge 500 with a hinge opening 510 and pins 520, a top jaw 600, a top jaw riser 610, and a bottom jaw 700, a bottom jaw riser 710, a top grip 800, a bottom grip 900 and the teeth 400.

The overall length of the clamp 100 may be between from about 5 mm to about 100 mm long. Preferably the length of the clamp is from about 25 mm to about 75 mm long, more preferably between 30 mm to about 65 mm long. In another preferred embodiment, the overall length of the clamp 100 is about 61 mm long. In a preferred embodiment, the overall clamp length is about 61 mm and the overall clamp width is from about 5.3 mm to about 9.4 mm. In a preferred embodiment, the overall clamp width tapers from about 5.3 mm to about 9.4 mm.

The length of the section of the vascular clamp 100 containing the teeth 400 is about from about 5 mm to about 100 mm long. Preferably the length of the teeth 400 is from about 25 mm to about 75 mm long, more preferably between 30 mm to about 65 mm long. In another preferred embodiment, the overall length of the teeth 400 is about 31 mm long.

In a preferred embodiment the overall length of the clamp is about 61 mm and the overall width is about 5.3 mm at the top and bottom jaws 600 and 700 sections, and the width is about 9.4 mm at the grip (800 and 900) sections.

In a preferred embodiment each top jaw riser 610 and the bottom jaw riser 710 is 5.3 mm in width and 31 mm in length, without the lengths of the hinge 500 and the top grip 800 or bottom grip 900.

The locking mechanism 250 consists of at least two hooks 200, a top grip 800, a bottom grip 900 and a receptacle 300. The receptacle 300 has an first opening 310 wider than the span of the hooks 210, and tapers to a second opening 320 narrower that the span of the hooks 210, and then widens a second time to a third opening 330 which is wider than the first opening 310, and is wider than the span of the hooks 210.

The receptacle 300 has a first opening 310, wherein the width of the first opening 310 is about 5.5 to about 10 mm. More preferably, the first opening 310 is greater than the span of the hinges 200. In a preferred embodiment, the width of first opening is greater than 8 mm. In a preferred embodiment, the width of first opening is about 8 mm.

The receptacle 300 also has a second opening 320 wherein the width of the second opening 320 is about 10 mm to about 20 mm across. In another embodiment the second opening 320 is about 2.5 to about 7.5 mm across. In a more preferred embodiment, the width of the second opening 320 is about 4.7 mm across.

The receptacle 300 also has a third opening 330, wherein the width of the third opening is about 10 mm to about 20 mm across. In another embodiment the third opening 330 is about 7.5 to about 15.5 mm across. In a more preferred embodiment, the width of third opening 330 is about 9.4 mm across.

The angle of the receptacle 300 opening, measured from its narrowest point of the receptacle 300 opening (the second opening 320) is about 75 degrees to about 150 degrees. In a preferred embodiment, the angle of the narrowest point of the receptacle opening (the second opening 320) is about 120 degrees.

The span of the hooks is from about 2 mm to about 10 mm, preferably 5.6 mm, more preferably 8 mm. Further, the span of the hooks 210 forms an angle from about 20 degrees when measured along the inner portion of the hooks 200 to about 120 degrees when measured along the outer portion of the hooks 200. Preferably the angle is about 40 degrees when measured along the inner portion of the hooks 200 to about 100 degrees when measured along the outer portion of the hooks 200.

In a preferred embodiment, the V-shaped hooks are at about a 40 degree angle on their top portion where they enter the receptacle 300.

The appearance of the top and bottom grips 800 and 900 may be varied. The top and bottom grips 800 and 900 have a rough surface to allow for a more secure grip by the user. In a preferred embodiment, the grips are grooves that are molded into the overall design. In an alternate embodiment, the grips can be covered in a rubber material to aid in gripping. In another embodiment, a tape may be applied which is a material of the type that aids in the gripping.

The shape of the top and bottom grips 800 and 900 may be varied. In one embodiment, they top grip 800 and the bottom grip 900 has a concave surface. The shape of the surface of the top grip 800 and the bottom grip 900 may be any size. The shape of the surface of the top grip 800 and the bottom grip 900 may be the same size. The shape of the surface of the top grip 800 and the bottom grip 900 may be different sizes.

In a preferred embodiment, the top grip 800 and the bottom grip 900 are each of equal lengths and widths. In a more preferred embodiment, the top grip and the bottom grip 800 and 900 are each about 9.4 mm in length and about 9.4 mm in width. In another embodiment, the top grip and said bottom grip are equal length and width. In another embodiment, top grip and said bottom grip are each about 9.4 mm in length and about 9.4 mm in width.

The vascular clamp 100 may be substantially straight without any pronounced curves or sectional bending. In a preferred embodiment, the top jaw 600 and the bottom jaw 700 have a top riser 610 and a bottom riser 710, respectively. The top riser 610 and the bottom riser 710 can be the same or different in size, length, width and shape. The top riser 610 and the bottom riser 710 may be from about 0.5 mm to about 5 mm in width, but is preferably about 2.4 mm in width. In another embodiment the top jaw riser and the bottom jaw riser each measure about 2.4 mm in width and about 31 mm in length

The present invention may be placed around an umbilical vascular or an umbilical vascular stump of an infant, and carry on all medically necessary functionality of a conventional clamp. Once the present invention is closed, it is permanently locked. Additionally, a transmitter within the sensor of sufficient strength may be able to communicate with a satellite receiver, a computer, a PDA or a smart phone. These types of sensors are well known in the art. There may also likely be some kind of interaction with a tracking technology, most likely a computer running a version of tracking software. The tracking technology may have a connection to the hospital's alarm or external speaker system or another type of warning or an alert implementation, which is triggered when a security incident is detected.

The preferred embodiment of the invention, with its accompanying dimensions, is described by FIGS. 9 and 10.

The invention, although useful for the human neonate, has other useful applications. It may be use to clamp any veins or arteries. Further, it may be applied in for use in non-human mammals.

The invention may also come in the form of a kit comprising the vascular clamp of described herein, an antiseptic, and a device to cut an umbilical cord.

Antiseptic may be selected from alcohols (ethanol (60-90%), 1-propanol (60-70%) and 2-propanol/isopropanol (70-80%) or mixtures of these alcohols. They are commonly referred to as “surgical alcohol”. Used to disinfect the skin before injections are given, often along with iodine (tincture of iodine), povidone-iodine; or some cationic surfactants (benzalkonium chloride 0.05-0.5%, chlorhexidine 0.2-4.0% or octenidine dihydrochloride 0.1-2.0%)), iodine as tincture of iodine or as Lugol's iodine, Quaternery ammonium compounds including benzalkonium chloride (BAC), cetyl trimethylammonium bromide (CTMB), cetylpyridinium chloride (Cetrim, CPC) and benzethonium chloride (BZT), and related disinfectants such as chlorhexidine and octenidine, Boric Acid, Brilliant Green, Chlorhexidine Gluconate, hydrogen peroxide; mercurochrome, manuka honey, octenidine dihydrochloride, phenol, hyperosmolar sodium chloride, sodium hypochlorite (with or without potassium permanganate), calcium hypochlorite, and terpines, such as tea tree oil.

The device to cut the umbilical cord may be selected from a sterile surgical blade, a sterile scissor, or any sharp preferably sterile device known to those skilled in the art.

Further, the older clamps are too small for physicians and nurses to handle, especially with gloves. Additionally, fluids accompanying the neonate during the birthing process such as mucus, amniotic fluid, merconium and blood, may cause the clamp to slip off the vascular or even out of the hands of the health care provider. This new invention solves the problem by increasing the surface area and adding additional angle of curvature at the grooves where the thumb and forefinger utilize the device. By widening the width of the finger grips the improved invention prevents the fingers from slipping off when closing the clamp which is the result of fluids making the gloves slippery.

The present invention is such that it solves these problems and also accommodates larger umbilical cords. Its larger teeth also grasp the umbilical cord better. Additionally, the invention optionally may be used with radio tags. Radio tags are utilized to alarm if the vascular clam opens, and may also be utilized to pin point the location of a neonate and prevent kidnapping. Further, radio tags can assist physicians in monitoring the neonates conditions by sending vital health information to a computer located at a nursing station. These signals can also be coordinated with smaller portable devices, such as PDAs and smart phones, which a doctor may keep with him or her at all times. The device offers the advantage over the prior art in that it has an increased length which accommodate any optional electronic devices. Furthermore, in instances where the umbilical cord is thick, this allows for easier closure of the device.

The inventions may be made from any material, but plastics, POM, acetal (POM) copolymer (Acetal (POM) Copolymer), polyvinyl chloride, polyethylene, polystyrene, polyvinyl chloride (PVC) polytetrafluoroethylene (PTFE), cellulose-based plastics, Bakelite, nylon, rubber, synthetic rubber and the like type of materials may be used. In a preferred embodiment, the vascular clamp is manufactured from POM. The invention may be manufactured in separate parts and joined together or may be made as one continuous instrument by injection molding techniques known to those skilled in the art. The invention may be colors coded to indicate a gender, a particular medical status, and the like.

Although this invention has been described with a certain degree of particularity, it is to be understood that the present disclosure has been made only by way of illustration and that numerous changes in the details of construction and arrangement of parts may be resorted to without departing from the spirit and the scope of the invention.

We claim:

1. A vascular clamp comprising: a clamp top, a clamp bottom, a locking mechanism, a top jaw, a top jaw riser, a bottom jaw, a bottom jaw riser, a top grip, a bottom grip, teeth and a hinge, wherein said top grip and said bottom grip are each about 9.4 mm in length and about 9.4 mm in width.

2. The vascular clamp of claim 1, wherein said top and said bottom are moveably connected by said hinge.

3. The vascular clamp of claim 2, wherein the hinge has an opening and a first pin extending from the bottom jaw and a second pin extending from the top jaw toward the opening, and said first and second pin being capable of popping the clamp open when the jaws are not pressed tightly to close the locking mechanism.

4. The vascular clamp of claim 1, wherein the overall clamp length is about 61 mm.

5. The vascular clamp of claim 1, wherein the overall clamp width tapers from about 5.3 mm to about 9.4 mm.

6. The locking mechanism of claim 1, wherein said locking mechanism comprises at least two hooks, a receptacle, a top grip and a bottom grip.

7. The hooks of claim 6, wherein said hooks form an angle of about 40 degrees when measured along their inner portion and about 100 degrees when measured along their outer portion.

8. The hooks of claim 7, wherein the span of the hooks is about 8 mm.

9. The vascular clamp of claim 1, wherein said top jaw and said bottom jaw measure about 31 mm in length.

10. The vascular clamp of claim 1, wherein said top grip and said bottom grip are each equal in length and width.

11. The top grip and the bottom grip of claim 6, wherein said top grip and said bottom grip are on a concave surface.

12. The vascular clamp of claim 1, wherein the overall length of the teeth is about 31 mm.

13. The receptacle of claim 1, wherein said receptacle has a first opening wider than the span of said hooks, and tapers to a second opening narrower that the span of the hooks, and then widens a second time to a third opening which is wider than the first opening and wider than the span of the hooks.

14. The receptacle of claim 13, wherein the width of the first opening is greater than about 8 mm, the width of the second opening is about 4.7 mm and the width of the third opening is about 9.4 mm.

15. The receptacle of claim 13, wherein said second opening has an angle of about 120 degrees.

16. The vascular clamp of claim 1, wherein said top jaw riser and said bottom jaw riser each measure about 2.4 mm in width and about 31 mm in length.

17. A kit comprising the vascular clamp of claim 1, an antiseptic, and a device to cut an umbilical cord.

18. A vascular clamp comprising a clamp top and a clamp bottom moveably connected by a hinge, a locking mechanism having at least two hooks, a top jaw, a top jaw riser, a bottom jaw, a bottom jaw riser, a top grip and a bottom grip on a concave surface, said top grip and said bottom grip being each about 9.4 mm in length and about 9.4 mm in width,
wherein the hinge has an opening and a first pin extending from the bottom jaw toward the opening and a second pin extending from the top jaw toward the opening, and said first and second pin being capable of popping the clamp open when the jaws are not pressed tightly to close the locking mechanism, and wherein the locking mechanism locks the clamp when the top and the bottom jaws are pressed tightly, and wherein the length of the clamp is about 61 mm.
